(19) United States
(12) Patent Application Publication
(10) Pub. No.: US 2015/0202308 A1
(43) Pub. Date: Jul. 23, 2015

(54) BUDESONIDE FORMULATION

(71) Applicant: Nephron Pharmaceuticals Corporation, Orlando, FL (US)

(72) Inventors: TRAVIS JOHN WEBB, Orlando, FL (US); ERIC PRIMELLES-PEREZ, Orlando, FL (US)

(73) Assignee: Nephron Pharmaceuticals Corporation, Orlando, FL (US)

(21) Appl. No.: 14/599,243

(22) Filed: Jan. 16, 2015

Related U.S. Application Data
(60) Provisional application No. 61/928,586, filed on Jan. 17, 2014.

(51) Int. Cl. A61K 47/48; A61K 31/58

(52) U.S. Cl. CPC A61K 47/4823; A61K 31/58

(57) ABSTRACT

The invention provides budesonide inhalation formulations containing cyclodextrins.

### RELATED APPLICATIONS

The present application claims priority under 35 U.S.C. §119(e) to U.S. provisional patent application, U.S. Ser. No. 61/928,586, filed Jan. 17, 2014, which is incorporated herein by reference.

### BACKGROUND OF INVENTION

Budesonide is a well-known anti-inflammatory corticosteroid that exhibits potent glucocorticoid activity. Budesonide is provided commercially as a mixture of two isomers (22R and 22S). Budesonide is indicated for maintenance and treatment of asthma and as prophylactic therapy in children.

Formulations of budesonide can be administered by inhalation using a nebulizer. Such formulations typically have been suspensions. In general, suspensions are believed to be less efficiently nebulized than solutions. Solutions of Budesonide are challenging to manufacture, as budesonide is insoluble in water. Budesonide solutions for nebulization are known. Such solutions have been prepared, in general, by the addition of a co-solvents or surfactants, many of which are undesirable. There is a recognized need for a budesonide solutions for administration via nebulization.

Saidi et al. (U.S. Pat. No. 6,241,969) disclose the preparation of corticosteroid-containing solutions for nasal and pulmonary delivery involving surfactants. Lintz et al. (AAPS Annual Meeting and Exposition, 2004) disclose the preparation of liquid formulations containing budesonide, water, citrate salt, sodium chloride and alcohol, propylene glycol and/or surfactant, such as Tween, Pluronic, or phospholipids with HLB-values between 10 and 20. Waldrep et al. (J. Aerosol Med. (1994), 7(2), 135-145) reportedly succeeded in preparing a liposome formulation of budesonide and phosphatidylcholine derivatives.

Cyclodextrins have been used to solubilize drugs. Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as .alpha.-, .beta.-, and .gamma.-cyclodextrin respectively. Each cyclodextrin subunit has secondary hydroxyl groups at the 2 and 3 positions and a primary hydroxyl group at the 6-position. The cyclodextrins may be pictured as hollow truncated cones with hydrophilic exterior surfaces and hydrophobic interior cavities. In aqueous solutions, these hydrophobic cavities provide a haven for hydrophobic organic compounds that can fit all or part of their structure into these cavities. This process, known as inclusion complexation, may result in increased apparent aqueous solubility and stability for the complexed drug. The so-called “inclusion complex” is stabilized by hydrophobic interactions and does not involve the formation of any covalent bonds.

The parent cyclodextrins often exhibit differing affinity for any given substrate. For example, .gamma.-cyclodextrin often forms complexes with limited solubility, resulting in solubility curves of the type Bs. This behavior is known for a large number of steroids which imposes serious limitations towards the use of gamma-cyclodextrins. Beta-cyclodextrins, however, do not complex well with a host of different classes of compounds. It has been shown for beta and gamma cyclodextrins that derivatization (e.g. alkylation) results in not only better aqueous solubility of the derivatives compared to the parent, but also changes the type of solubility curves from the limiting B-type to the more linear A-type curve (Bernd W. Muller and Ulrich Brauns, “Change of Phase-Solubility Behavior by Gamma-Cyclodextrin Derivatization”, Pharmaceutical Research (1985) p 309-310.

Chemical modification of the parent cyclodextrins (usually at the hydroxyls) has resulted in derivatives with improved safety while retaining or improving the complexation ability. Of the numerous derivatized cyclodextrins prepared to date, only two appear to be commercially viable: the 2-hydroxypropyl derivatives (HP-CD; neutral cyclodextrins being commercially developed by Janssen and others), and the sulfoalkyl ether derivatives, such as sulfobutylether (SBE-CD; anionic cyclodextrins being developed by CyDex, Inc.)

A number of studies regarding the use of cyclodextrins for inhalation have been reported, although none have been commercialized. The studies suggest that different drug-cyclodextrin combinations will be required for specific optimal or even useful inhaled or intra-nasal formulations. In almost every case, solvents, solubilizing polymers and other ancillary agents, all of which are generally undesirable, are employed to permit adequate solubilization of the cyclodextrin and formation of desired inclusion complex.

Cyclodextrins have been proposed to solubilize steroids. U.S. Pat. No. 4,383,992 discloses that beta-cyclodextrins can form inclusion complexes with corticosteroids. Molar ratios of 1:1 cyclodextrin:steroid are proposed, and “dispersing agents' such as hydroxypropylmethylcellulose are proposed to facilitate dissolution of the cyclodextrin.

Cyclodextrins have been proposed to solubilize budesonide. U.S. Pat. No. 5,914,122 to Otterbeck et al. discloses a budesonide preparation. Otterbeck teaches that budesonide is stabilized with low pH. The budesonide can be combined with any number of ancillary agents and solubilizers, including thickeners, co-solvents, and cyclodextrins. The examples show combinations including cyclodextrins (in molar ratio to budesonide of about 30:1) dissolved in ethanol (400 mg) water (60 mg), together with a thickener (xanthum gum) and a preservative (sodium benzoate).

Cyclodextrins also have been proposed for solubilizing drugs where the solubilizing solution contains a drug, the cyclodextrin, and either an ‘accompanying ‘guest’ molecule and/or solubilizing polymer such as a cellulose derivatives (e.g., hydroxypropylmethylcellulose), a vinyl derivatives (e.g., polyvinyl alcohol), acrylic acid polymers and the like. See U.S. Pat. No. 7,115,586, the disclosure of which is incorporated herein by reference.

Another example of cyclodextrin combined with budesonide is shown in U.S. Publication 2006/0193783. The examples show the combination was always in the presence of a solubilizing agent such as hydroxypropyl methylcellulose and N-methyl pyrollidone. The molar ratios of cyclodextrin:budesonide did not exceed about 25:1.

Another example of cyclodextrin combined with budesonide is shown in U.S. Publication 2007/0020196. This application includes an extensive discussion of the history of cyclodextrins and is incorporated herein in its entirety by reference. This application purports to discover that sulfoalkyl ether cyclodextrins (SAE cyclodextrin) are particularly suitable for inhalable solutions of budesonide. SAE cyclodextrins are a more soluble form of cyclodextrin than beta or gamma cyclodextrins.

In summary, the art suggests that, in some cases, solutions may be preferred over suspensions for nasal and pulmonary delivery. Even though the art discloses inhalable solutions containing a corticosteroid and cyclodextrin, the results of the art demonstrate unpredictability. The combination of one cyclodextrin with one drug does not necessarily suggest that another cyclodextrin will be suitable.

A need remains in the art for a stabilized aqueous budesonide solution that does not require the addition of preservatives, surfactants and/or co-solvents. A need also remains for a manufacturing protocol that permits fast and reliable manufacture of such a solution.

Other challenges in preparing Inhalation preparations of budesonide include making sure that the final composition contains appropriate levels of the two epimers of budesonide. The epimers establish themselves at different levels in different circumstances. Another challenge is making sure that there is not unacceptably high levels of uncomplexed cyclodextrins in the final product. Another challenge is making sure that the final preparation has an appropriate pharmaceutical dose of budesonide. Another challenge is avoiding unnecessary loss of budesonide in the manufacture process.

### SUMMARY OF THE INVENTION

It has been discovered, unexpectedly, that budesonide can be complexed with beta and gamma cyclodextrins under conditions leaving little budesonide uncomplexed, thereby avoiding loss of drug compound.

It has been discovered, unexpectedly, that budesonide can be complexed with beta and gamma cyclodextrins in minutes, and in a reproducible manner, using very simply parameters.

It has been discovered, unexpectedly, that stable complexes of budesonide and beta and gamma cyclodextrins can be manufactured quickly and efficiently, without co-solvents, surfactants, polymer stabilizing agents and preservatives—any one, combination or all of which may be undesirable.

In one aspect of the invention, it was discovered that an ionic solution will facilitate complexing of budesonide and cyclodextrins. A method is provided for preparing a pharmaceutical product. The method involves forming an aqueous complexing solution having an osmolality of at least 400 mOsm/Kg or an ionic strength of at least 290 mol/m⁻³ and containing cyclodextrin and budesonide, the cyclodextrin and budesonide capable of forming a cyclodextrin-budesonide inclusion complex, permitting the cyclodextrin and budesonide inclusion complex to form, and then diluting the complexing solution to provide the pharmaceutical composition having an osmolality of between 260 mOsm/kg and 330 mOsm/kg. In embodiments, the osmolality of the complexing solution is at least: 400 mOsm/kg, 600 mOsm/kg, 900 mOsm/kg, 1200 mOsm/kg, 1500 mOsm/kg, 1800 mOsm/kg, 2100 mOsm/kg, 2400 mOsm/kg, 2700 mOsm/kg, 3000 mOsm/kg, or 3500 mOsm/kg. In embodiments, the ionic strength of the complexing solution is at least: 290 mol/m⁻³, 435 mol/m⁻³, 650 mol/m⁻³, 870 mol/m⁻³, 1090 mol/m⁻³, 1200 mol/m⁻³, 1400 mol/m⁻³ or 1500 mol/m⁻³. Thus, a first solution which is an ionic and not pharmaceutically acceptable is prepared to assist in forming the inclusion complex, and then that solution is diluted to an osmolality which is pharmaceutically acceptable, substantially without loss of the inclusion complex formed. The complexing can be achieved very quickly, in some embodiments with more than 99% efficiency in less than 2 hours, less than 1 hour, less than 45 minutes, less than 30 minutes, less than 20 minutes, and even less than 10 minutes.

In embodiments, the molar ratio of cyclodextrin to budesonide in the complexing solution can be between 20:1 and 80:1. In embodiments, molar ratio of cyclodextrin to budesonide in the complexing solution can be between 40:1 and 60:1. In embodiments, the molar ratio of cyclodextrin to budesonide in the complexing solution can be at least 45:1, at least 50:1, at least 55:1, or at least 60:1.

In any of the foregoing embodiments, the complexing solution preferably can be 60%-100% cyclodextrin saturated solution. In any of the foregoing embodiments, the complexing solution can be a 90%-100% cyclodextrin saturated solution.

In any of the foregoing embodiments, the pH of the complexing solution is below 6, or between 3.5 and 4.5. In any of the foregoing embodiments, the complexing solution may contain any one or more of NaCl, a buffer and EDTA. In some embodiments, the complexing solution contains NaCl, a buffer and EDTA.

In any of the foregoing embodiments, the aqueous complexing solution can be formed by first mixing the cyclodextrin as a solid with the budesonide as a solid to form a mixture of solids, and then contacting the mixture of solids with an ionic aqueous solubilizing solution to form the complexing solution. In embodiments, the ionic aqueous solubilizing solution is at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³. In embodiments, the ionic aqueous solubilizing solution may contain any one or more of NaCl, a buffer and EDTA. In some embodiments, the ionic aqueous solubilizing solution contains NaCl, a buffer and EDTA.

In some embodiments, the complexing solution is contacted with a pH adjusting agent to adjust the pH of the complexing solution to below 6 or to between 3.5 and 4.5.

In any of the foregoing embodiments, the cyclodextrin preferably can be a beta or gamma cyclodextrin. In any of the foregoing embodiments, the cyclodextrin preferably can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

In any of the foregoing embodiments, the complexing can occur in the absence of any one, absence of any combination of or absence of all of (i) a co-solvent, (ii) sodium benzoate or any preservative other than citric acid and EDTA, (iii) a stabilizing polymer, and (iv) a thickener.

In another aspect of the invention, it was discovered that stable pharmaceutical preparations of budesonide and cyclodextrins can be prepared using a very high molar ratio of cyclodextrin to budesonide, with subsequent dilution to achieve a stable pharmaceutical solution of budesonide containing a desired amount of budesonide and acceptable levels of cyclodextrins. A method of preparing a pharmaceutical product is provided. The method involves forming an aqueous complexing solution and containing cyclodextrin and budesonide, the cyclodextrin and budesonide capable of forming a cyclodextrin-budesonide inclusion complex, wherein the molar ratio of cyclodextrin to budesonide in the complexing solution is greater than 40:1, permitting the cyclodextrin and budesonide inclusion complex to form, and diluting the complexing solution to provide a pharmaceutical composition, wherein the pharmaceutical composition has a pH of less than 6.0 and an osmolality of between 260 mOsm/kg and 330 mOsm/kg. In embodiments, the molar ratio of cyclodextrin to budesonide in the complexing solution can be greater than 50:1. In embodiments, the molar ratio of cyclodextrin to budesonide in the complexing solution can be greater than 55:1, or greater than 60:1. In embodiments, molar ratio of cyclodextrin to budesonide in the complexing solution can be between 45:1 and 100:1. Thus, a first solution which is not pharmaceutically acceptable is prepared to assist in forming the inclusion complex, and then that solution is diluted to form a solution that is pharmaceutically acceptable, substantially without loss of the inclusion complex formed. The complexing can be achieved very quickly, in some embodiments with more than 99% efficiency in less than 2 hours, less than 1 hour, less than 45 minutes, less than 30 minutes, less than 20 minutes, and even less than 10 minutes.

In any of the foregoing embodiments, the osmolality of the complexing solution can be at least: 400 mOsm/kg, 600 mOsm, 900 mOsm/kg, 1200 mOsm/kg, 1500 mOsm/kg, 1800 mOsm/kg, 2100 mOsm/kg, 2400 mOsm/kg, 2700 mOsm/kg, 3000 mOsm/kg, or 3500 mOsm/kg. In some embodiments, the osmolality of the complexing solution is between 400 mOsm/kg and 3500 mOsm/kg. In some embodiments, the osmolality of the complexing solution is between 800 mOsm/kg and 3500 mOsm/kg. In any of the foregoing embodiments, the ionic strength of the complexing solution can be at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³. In some embodiments, the ionic strength of the complexing solution is between 290 mol/m⁻³ and 1500 mol/m⁻³. In some embodiments, the ionic strength of the complexing solution is between 650 mol/m⁻³ and 1500 mol/m⁻³.

In order to favorably affect the reaction kinetics and reduce the amount of cyclodextrins in the final solution while efficiently and reproducibly forming inclusion complexes, the complexing solution in embodiments can be, for example, a 60%-100% cyclodextrin saturated solution. In embodiments, the complexing solution is a 90%-100% cyclodextrin saturated solution.

In any of the foregoing embodiments, the aqueous complexing solution can be formed by first mixing the cyclodextrin as a solid with the budesonide as a solid to form a mixture of solids, and then contacting the mixture of solids with an aqueous solubilizing solution to form the complexing solution.

In embodiments, the ionic strength of the aqueous solubilizing solution can be at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³. In some embodiments, the ionic strength of the aqueous solubilizing solution is between 290 mol/m⁻³ and 1500 mol/m⁻³. In some embodiments, the ionic strength of the aqueous solubilizing solution is between 650 mol/m⁻³ and 1500 mol/m⁻³.

In embodiments, the ionic aqueous solubilizing solution may contain any one or more of NaCl, a buffer and EDTA. In some embodiments, the ionic aqueous solubilizing solution contains NaCl, a buffer and EDTA.

In any of the foregoing embodiments, the complexing solution can be contacted with a pH adjusting agent to adjust the pH of the complexing solution to below 6 or to between 3.5 and 4.5.

In any of the foregoing embodiments, the cyclodextrin preferably can be a beta or gamma cyclodextrin. In any of the foregoing embodiments, the cyclodextrin preferably can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

In any of the foregoing embodiments, the complexing can occur in the absence of any one, absence of any combination of or absence of all of (i) a co-solvent, (ii) sodium benzoate or any preservative other than citric acid and EDTA, (iii) a stabilizing polymer, and (iv) a thickener.

In any of the foregoing embodiments, the budesonide is present in the complexing solution at a concentration of between 0.01 mg/mL and 7.5 mg/mL.

In any of the foregoing embodiments, the budesonide is present in the pharmaceutical composition at a concentration of between 0.001 mg/mL and 0.75 mg/mL.

In any of the foregoing embodiments, the budesonide is present in the pharmaceutical composition at a concentration of between 0.09 mg/mL and 0.50 mg/mL.

In any of the foregoing embodiments, the budesonide is present in the pharmaceutical composition at a concentration of between 0.10 mg/mL and 0.25 mg/mL.

According to another aspect of the invention, a composition is provided. The composition is an aqueous solution having an osmolality of at least 400 mOsm/kg or an ionic strength of at least 290 mol/m⁻³ and containing a cyclodextrin and budesonide, wherein at least 95%, at least 96%, at least 97%, at least 98%, or even at least 99% of the budesonide in the solution is complexed with cyclodextrin, and wherein the aqueous solution is free of any one of, any combination of, or all of (i) a co-solvent (ii) sodium benzoate or any preservative other than citric acid and EDTA, (iii) a stabilizing polymer, and (iv) a thickener. In embodiments, the molar ratio of cyclodextrin to budesonide is at least 40:1, at least 45:1, at least 50:1, at least 55:1, at least 60:1, or at least 75:1. In embodiments, the molar ratio of cyclodextrin to budesonide is between 45:1 and 100:1.

In some embodiments, the osmolality of the complexing solution is between 400 mOsm/kg and 3500 mOsm/kg. In some embodiments, the ionic strength of the complexing solution is between 290 mol/m⁻³ and 1500 mol/m⁻³.

In any of the foregoing embodiments, the cyclodextrin preferably can be a beta or gamma cyclodextrin. In any of the foregoing embodiments, the cyclodextrin preferably can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

According to another aspect of the invention, a composition is provided. The composition is a dry mixture of a cyclodextrin and budesonide, wherein the molar ratio of cyclodextrin to budesonide is at least 40:1, at least 45:1, at least 50:1, at least 55:1, at least 60:1, or at least 75:1. In embodiments, the molar ratio of cyclodextrin to budesonide is between 45:1 and 100:1. In any of the foregoing embodiments, the cyclodextrin preferably can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

According to another aspect of the invention, a pharmaceutical composition is provided. The pharmaceutical composition is an aqueous solution having an osmolality of between 260 mOsm/kg and 330 mOsm/kg, wherein the solution contains cyclodextrin and budesonide and EDTA, wherein the cyclodextrin and budesonide are in molar ratio of at least 40:1, at least 45:1, at least 50:1, at least 55:1, at least 60:1, or at least 75:1, wherein the budesonide is present in a concentration of between 0.001 mg/mL and 0.75 mg/mL, and wherein at least 95% of the budesonide in the solution is complexed with cyclodextrin. Preferably, the aqueous solution is a buffered aqueous solution. In some embodiments, the aqueous solution further comprises a citrate buffer, and sodium chloride. In any of the foregoing embodiments, the aqueous solution can be free of any one, any combination of or all of (i) a co-solvent, (ii) sodium benzoate or any preservative other than citric acid and EDTA, (iii) a stabilizing polymer, and (iv) a thickener.

In any of the foregoing embodiments, the cyclodextrin preferably can be a beta or gamma cyclodextrin. In any of the foregoing embodiments, the cyclodextrin preferably can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

In any of the foregoing embodiments, the budesonide can be present in the pharmaceutical composition at a concentration of between 0.05 mg/mL and 0.60 mg/mL, 0.09 mg/mL and 0.50 mg/mL or 0.10 mg/mL and 0.25 mg/mL.

To promote the complexation between budesonide and cyclodextrin, budesonide (e.g., budesonide prior to the complexation with cyclodextrin) may be in the form of particles. To achieve at least 95%, at least 96%, at least 97%, at least 98%, at least 99%, or 100% complexation between budesonide and cyclodextrin, the size of the budesonide particles may be less than or equal to 50, less than or equal to 40, less than or equal to 35, less than or equal to 30, or less than or equal to 25 μm.

Another aspect of the invention relates to compositions and pharmaceutical products (e.g., pharmaceutical compositions) prepared by a method described herein.

According to another aspect of the invention, a pharmaceutical composition is provided. The composition is a solution consisting of a cyclodextrin, budesonide, NaCl, EDTA, a buffer and water. The osmolality preferably is between 260 mOsm/kg and 330 mOsm/kg. In embodiments, the molar ratio of cyclodextrin to budesonide can be at least 40:1, at least 45:1, at least 50:1, at least 55:1, at least 60:1, or at least 75:1. In embodiments, the molar ratio of cyclodextrin to budesonide is between 45:1 and 100:1. In embodiments, the budesonide is present in a concentration of between 0.001 mg/mL and 0.75 mg/mL. In embodiments, at least 95%, at least 96%, at least 97%, at least 98%, or even at least 99% of the budesonide in the composition is complexed with cyclodextrin. In embodiments, the pH of the pharmaceutical composition is below 6. In embodiments, the pH is between 3.5 and 4.5. In any of the foregoing embodiments, the cyclodextrin preferably can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

In any of the foregoing embodiments, the budesonide can be present in the pharmaceutical composition at a concentration of between 0.05 mg/mL and 0.60 mg/mL, 0.09 mg/mL and 0.50 mg/mL or 0.10 mg/mL and 0.25 mg/mL.

According to another aspect of the invention, a method of treatment is provided. The method involved administering to a subject in need of such treatment an effective amount of any one of the pharmaceutical compositions described above. Subjects, conditions, symptoms and treatments are described below, as if fully recited in this summary of invention.

### BRIEF DESCRIPTION OF THE DRAWINGS

FIG. 1 is a graph showing the relating rate of budesonide complexation in buffer vs. water.

### DETAILED DESCRIPTION

Budesonide has the following chemical formula: 16,17-(butylidenebis(oxy))-11,21-dihydroxy-, (11-β,16-α)-pregna-1,4-diene-3,20-dione. It has the chemical structure:

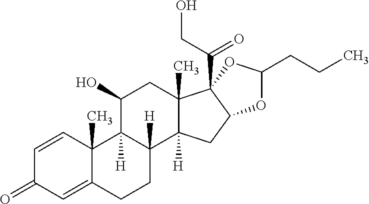

Budesonide is typically provided as a mixture of two epimers (22R and 22S). The two forms do not interconvert. The 22R epimer is more active than the 22S epimer.

Cyclodextrins are described above and also are disclosed, for example, in U.S. Pat. Nos. 4,383,992, 5,914,122, and 7,115,586, the entire disclosures of which are incorporated herein by reference. Cyclodextrins are also described in U.S. Patent Applications Pub No. 2006/0193783 and 2007/0020196, the entire disclosures of which are incorporated herein by reference. In any the embodiments described herein, the cyclodextrin preferably can be a beta or gamma cyclodextrin. In any of the embodiments described herein, the cyclodextrin can be 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

A cyclodextrin-budesonide inclusion complex is a complex in which the cyclodextrin (the “host”) forms a cavity in which the molecule of budesonide (the “guest”) is positioned in whole or in part.

A solubilizing solution is prepared for combining with the solid mixture of the budesonide and the cyclodextrin. The solubilizing solution is combined with the solid mixture of budesonide and cyclodextrin to form the complexing solution. The solubilizing solution is typically prepared to be strongly ionic, such that the solid mixture is immediately introduced into an environment having the appropriate ionic strength. The solubilizing solution may contain, in addition to the elements establishing the appropriate ionic strength, other materials that will be found in the final pharmaceutical preparation, such as a chelating agent (for example, EDTA) and a buffer.

In one embodiment, the cyclodextrin and solubilizing solution are in relative amounts such that the combination to form the complexing solution forms a saturated cyclodextrin solution. A saturated solution is the point at which no more of a substance can dissolve and additional amounts of the substance will appear as a separate phase and not go into solution. It will be understood by one of ordinary skill in the art that the presence of other substances in the complexing solution will affect the degree to which cyclodextrin can be solubilized. In some embodiments, the complexing solution is at least 60%, at least 70%, at least 80%, at least 90%, at least 95%, or at least 99% cyclodextrin saturated.

The complexing solution is the solution in which the budesonide and the cyclodextrin are combined and mixed for forming the budesonide-cyclodextrin inclusion complexes. According to the present invention, the complexing solution is a strong ionic solution, which facilitates the displacement of water in the cyclodextrin core with budesonide. Surprisingly, the invention permits substantially all of the budesonide in the complexing solution to combine with cyclodextrin, and to do so rapidly. In some embodiments, substantially all means at least 95%, at least 96%, at least 97%, at least 98%, or at least 99% of the budesonide in the complexing solution is part of an inclusion complex. For example, substantially all of the budesonide in the complexing solution combines with cyclodextrin in less than 120 minutes, less than 60 minutes, less than 30 minutes, less than 20 minutes, and even less than 10 minutes. In addition, under these conditions, the relative amounts of the two budesonide epimers in the inclusion complexes are substantially equal. Maintaining a predictable relative amount of the epimers under manufacturing conditions is important for meeting regulatory requirements, and it was surprising that the epimers loaded so rapidly in approximately equal amounts.

The invention does not require the presence of co-solvents to facilitate the formation of the inclusion complexes. Thus, unnecessary and undesirable co-solvents can be avoided. Thus, the complexation solution, according to an aspect of the invention, can be free of one or more of, or all of, alcoholic co-solvents and other non-aqueous co-solvents such as ethanol, glycerol, propylene glycol, polyethylene glycol, polyhydric alcohol, triethylene glycol and poloxamer.

The invention also does not require the presence of complexation-enhancing agents such as solubilizing polymers and surfactants that facilitate the formation of the inclusion complexes. Unnecessary and undesirable materials can be avoided. Thus, the complexation solution, according to an aspect of the invention, can be free of one or more of, or all of, polymers and surfactants such as cellulose and cellulose derivatives, N-methyl-pyrrolidone, vinyl/poly vinyl pyrrolidone polymers, polyvinyl alcohol or mixtures thereof. Other examples of complex enhancing agents include pharmacologically inert water soluble polymers, hydroxy acids, and other organic compounds typically used in liquid formulations to enhance the complexation of a particular agent with cyclodextrins. The natural polymers include polysaccharides such as inulin, pectin, algin derivatives (e.g. sodium alginate) and agar, and polypeptides such as casein and gelatin. The semi-synthetic polymers include cellulose derivatives such as methylcellulose, hydroxyethylcellulose, hydroxypropyl cellulose, their mixed ethers such as hydroxypropyl methylcellulose and other mixed ethers such as hydroxyethyl ethylcellulose and hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer).

The complexing solution and the pharmaceutical composition, according to an aspect of the invention, are free of preservatives other than EDTA and citric acid.

The complexing solution, according to an aspect of the invention, is free of thickening agents Thickening agents non-exclusively include hydroxy alkyl alky celluloses such as hydroxy propyl methyl cellulose, hydroxylethyl cellulose, hydroxyl methyl cellulose; carboxy alkyl celluloses and their salts such as sodium carboxy methyl cellulose; methyl cellulose; polysaccharides such as alginic acid, agar, guar gum, xanthan gum; polyacrylic acids such as polymethacrylic acid derivatives; polyvinyl pyrrolidone, maltodextrines.

Buffer.

A buffer is either a weak acid and its salt or a weak base and its salt, which in solution resists potential changes in pH. The solutions of the invention can include a buffer. In any of the embodiments, the buffer can be disodium phosphate and Phosphoric acid. Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof. Citric acid is stated in some references to have buffering properties. Thus, in the context of a present invention, when a solution is free of a buffer, it is meant that the solution is free of a buffer other than citric acid. For example, a solution containing both citric acid and sodium citrate is a buffered solution, and such a solution is not free of a buffer other than citric acid. Whereas a solution containing only citric acid and not a salt such as sodium citrate, is a solution free of a buffer other than citric acid.

Chelating Agent.

A chelating agent is a ligand that can form a chelate with a metal atom. Chelation involves the formation or presence of two or more separate coordinate bonds between a polydentate (multiple bonded) ligand and a single central atom. Well known chelating agents include EDTA, that is edetic acid and edetic acid salts like disodium edetate, sodium edetate, edetate calcium disodium and trisodium edetate, malic acid and mixtures thereof. Citric acid is stated in some references to be a chelating agent. In some embodiments, the solutions of the invention contain one or both of citric acid and edetate. In other embodiments, the solutions of the invention can be free of one or both of citric acid and edentate disodium or free of any chelating agent.

Antioxidant.

An antioxidant is a molecule that inhibits the oxidation of other molecules. In the context of the present invention, an antioxidant is one known to inhibit the oxidation of other molecules in an aqueous solution. Citric acid and edentate disodium are stated in some references to have anti-oxidant properties. In some embodiments, the solutions of the invention contain one or both of citric acid and edetate. In other embodiments, the solutions of the invention can be free of one or both of citric acid and edentate disodium or free of any anti-oxidant.

The solutions of the invention can be free of the preservative benzalkonium chloride. The solutions can be free of polymeric quaternary ammonium compounds that are preservatives. The solutions can be free of any preservative other than a chelating agent. The solutions can be free of any preservative, including free of chelating agents. Exemplary preservatives include antioxidants, chelating agents, antimicrobial preservatives, antifungal preservatives, alcohol preservatives, and acidic preservatives. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, sodium sulfite and vitamin E polyethylene glycol succinate. Exemplary antimicrobial preservatives include benzalkonium chloride, benzethonium chloride, benzyl alcohol, boric acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, and potassium metabisulfite.

Size of Budesonide Particles.

To promote the complexation between budesonide and cyclodextrin, budesonide (e.g., budesonide prior to the complexation with cyclodextrin) may be in the form of particles. In certain embodiments, the size of a budesonide particle (particle size of budesonide) described herein refers to the Feret diameter (e.g., minimum Feret diameter) of the budesonide particle. In certain embodiments, the size of the budesonide particles is the size obtained by sieve analysis of the budesonide particles. In certain embodiments, the size of the budesonide particles is an average (e.g., number average) of the sizes of the budesonide particles. In certain embodiments, the size of the budesonide particles is the largest of the sizes of the budesonide particles. In certain embodiments, the size of the budesonide particles is less than or equal to 100, less than or equal to 80, less than or equal to 60, less than or equal to 50, less than or equal to 40, less than or equal to 35, less than or equal to 30, less than or equal to 25, less than or equal to 20, less than or equal to 15, or less than or equal to 10 μm. In certain embodiments, the size of the budesonide particles is at least 30, at least 25, at least 20, at least 15, at least 10, at least 3, at least 1, at least 0.1, at least 0.01, or at least 0.001 μm. Any and all combinations of the ranges described herein (e.g., less than or equal to 35 μm and at least 0.1 μm (between 0.1 and 35 μm, inclusive)) are also within the scope of the invention. In certain embodiments, the size of the budesonide particles is less than or equal to 50 μm. In certain embodiments, the size of the budesonide particles is less than or equal to 40 μm. In certain embodiments, the size of the budesonide particles is less than or equal to 35 μm. In certain embodiments, the size of the budesonide particles is less than or equal to 30 μm. In certain embodiments, the size of the budesonide particles is less than or equal to 25 μm. In certain embodiments, the sizes of at least 90% of the budesonide particles are between 0.01 and 50, between 0.1 and 50, between 1 and 50, or between 10 and 50 μm, inclusive. In certain embodiments, the sizes of at least 90% of the budesonide particles are between 0.01 and 40, between 0.1 and 40, between 1 and 40, or between 10 and 40 μm, inclusive. In certain embodiments, the sizes of at least 90% of the budesonide particles are between 0.01 and 35, between 0.1 and 35, between 1 and 35, or between 10 and 35 μm, inclusive. In certain embodiments, the sizes of at least 90% of the budesonide particles are between 0.01 and 30, between 0.1 and 30, between 1 and 30, or between 10 and 30 μm, inclusive. In certain embodiments, the sizes of at least 90% of the budesonide particles are between 0.01 and 25, between 0.1 and 25, between 1 and 25, or between 10 and 25 μm, inclusive. In certain embodiments, at least 95%, at least 96%, at least 97%, at least 98%, or at least 99% complexation between budesonide and cyclodextrin is achieved when the budesonide is in the form of particles and when the size of the budesonide particles is as described herein. In certain embodiments, 100% complexation between budesonide and cyclodextrin is achieved when the budesonide is in the form of particles and when the size of the budesonide particles is as described herein.

Another aspect of the invention relates to compositions and pharmaceutical products (e.g., pharmaceutical compositions) prepared by a method described herein.

The solutions of the invention can be used to treat a subject with an allergic condition. “Treat”, “treating” and “treatment” encompass an action that occurs while a subject is suffering from a condition which reduces the severity of the condition (or a symptom associated with the condition) or retards or slows the progression of the condition (or a symptom associated with the condition). This is therapeutic treatment. “Treat”, “treating” and “treatment” also encompasses an action that occurs before a subject begins to suffer from the condition (or a symptom associated with the condition) and which inhibits the onset of or reduces the severity of the condition (or a symptom associated with the condition). This is prophylactic treatment.

Subjects are treated with effective amounts of the solutions of the invention. An “effective amount” of a compound generally refers to an amount sufficient to elicit the desired biological response, i.e., treat the condition. As will be appreciated by those of ordinary skill in this art, the effective amount of a compound described herein may vary depending on such factors as the condition being treated, the mode of administration, and the age and health of the subject. The condition treated by the solutions of the invention can be an allergic condition manifested by inflammation, itchy nose, itchy mouth, itchy eyes, itchy throat, runny nose, sneezing, watery eyes, and/or hyper-reactivity of the airways. An effective amount encompasses therapeutic and prophylactic treatment.

For therapeutic treatment, an effective amount is an amount sufficient to provide a therapeutic benefit in the treatment of a condition or to reduce or eliminate one or more symptoms associated with the condition. This may encompass an amount that improves overall therapy, reduces or avoids symptoms or causes of the condition, or enhances the therapeutic efficacy of another therapeutic agent.

For prophylactic treatment, an effective amount is an amount sufficient to prevent, delay the onset of, or reduce the severity of a condition, or one or more symptoms associated with the condition, or prevent its recurrence. This may encompass an amount that improves overall prophylaxis or enhances the prophylactic efficacy of another prophylactic agent.

A subject as used herein means a human.

Administering as used herein means contacting affected tissue of the subject, for example by topically applying eye drops to the eye.

The inhalation formulation is used for the treatment of asthma, non-infectious rhinitis (including hay fever and other allergies), and for treatment and prevention of nasal polyposis.

The pathophysiology of asthma and related disorders involves various symptoms, including bronchoconstriction, inflammation of the airways, and increased mucous secretion, which results in wheezing, coughing and shortness of breath. A persistent or recurrent cough may exacerbate the problem by causing further irritation and inflammation of the airways. Bronchoconstriction occurs due to bronchial smooth muscle spasm and airway inflammation with mucosal edema.

The invention includes methods for treatment, prevention, or amelioration of one or more symptoms of bronchoconstrictive disorders. Bronchoconstrictive disorders,” as used herein, refers to any disease or condition which can be physically manifested by the constriction or narrowing of the bronchi. Examples of bronchoconstrictive disorders include, but are not limited to, asthma, pediatric asthma, bronchial asthma, allergic asthma, intrinsic asthma, chronic obstructive pulmonary disease (COPD), chronic bronchitis, and emphysema.

A formulation, according to aspects of the invention, will have a storage shelf life of no less than 6 months. In this case, shelf life is determined only as regards the increase in the amount of budesonide degradation by-products or a reduction in the amount of budesonide remaining in the formulation. For example, for a formulation having a shelf life of at least six months, the formulation will not demonstrate an unacceptable and substantial increase in the amount of degradants during the storage period of at least six months. The criteria for acceptable shelf-life are set as needed according to a given product and its storage stability requirements. In other words, the amount of degradants in a formulation having an acceptable shelf-life will not increase beyond a predetermined value during the intended period of storage. On the other hand, the amount of degradants of a formulation having an unacceptable shelf-life will increase beyond the predetermined value during the intended period of storage.

### EXAMPLES

The rate and efficiency of the complexation process of a drug with a cyclodextrin is, in most cases, the limiting factor for the usefulness of the cyclodextrin as a solubilizing agent for the drug. Complexation of budesonide with cyclodextrins can take hours to days and, even then, is often in-efficient in maximally complexing the available budesonide with cyclodextrin.

Traditional methods for complexation include dry mixing in a mill which requires significant physical force to achieve complexation; or mixing as a slightly wetted paste which requires less force but operates under the same general principle. There is also wet mixing in water which in the case of highly insoluble molecules like Budesonide may be in-effective to achieve complexation. All of these methods require hours or days to effectively complex budesonide with HP-β-CD, and typically have yields of 50%-80% efficiency for liquid preparations with no organic solvents.

The invention involves the discovery of a budesonide inhalation solution, made using a strongly ionic, cyclodextrin-saturated, complexation solution. The method utilizes a high concentration buffer solution that catalyzes the rapid and complete complexation of budesonide and cyclodextrin.

In the first step of this procedure, budesonide and cyclodextrin are mixed together dry. This dry mixing of components produces a uniform distribution to help avoid aggregation of budesonide, which is highly hydrophobic and tends to agglomerate and float on the surface of water. Such aggregation would reduce the efficiency of the complexation.

In the second step, a small amount of concentrated buffer solution is introduced to the dry mixture to create an ionic solution saturated with the dry mixture, and particularly saturated with cyclodextrin which is in molar excess. The saturation of the solution helps to prevent budesonide from migrating to the surface and the high concentration of salts creates a favorable thermodynamic gradient for the complexation reaction. Strong ions such as Sodium, Chloride, and Citrate do not interact to a significant degree with the hydrophobic core of the cyclodextrin and facilitate the displacement of water molecules form the core through an osmotic gradient. When the water is displaced from the core it catalyzes the complexation of budesonide with the cyclodextrin.

### Example 1

Studies were conducted to determine the minimum concentration of 2-Hy-B-cyclodextrin required to achieve 100% budesonide complexation. It was demonstrated the 4% (w/v) beta-cyclodextrin was sufficient to achieve stable 100% complexation of Budesonide at 0.188 mg/mL. To evaluate if a lower concentration may be used a study was conducted to evaluate if 100% complexation was possible at between 0.5% and 3.0% cyclodextrin.
Complexation efficiency in buffer vs. water: Traditional methods of complexation are usually carried out in water. We conducted studies to evaluate the efficiency of Budesonide-cyclodextrin complexation using a complexation solution of high ionic strength and using purified water.
Alternative Salts or Complexation: The ionic strength of the Buffer solution was calculated to be approximately 508 mol/m⁻³. Two alternative salts, Sodium Chloride and Potassium Chloride, and a Phosphate buffer were prepared at the same ionic strength. Laboratory batches of budesonide inhalation solution were prepared using each alternative ionic adjuster to evaluate the effects of various salts on the formulation process.
Alternative Beta-Cyclodextrins for Complexation: Three additional Beta Cyclodextrins, 2-hydroxyethyl-B-cyclodextrin and Heptakis 2,6-Di-O-Methyl-B-cyclodextrin, were evaluated as potential alternatives for complexation with Budesonide.

Materials: Budesonide API Material, Farmabios; Citric Acid, Anhydrous, EMD; Sodium Citrate, Dihydrate, J. T. Baker; Sodium Chloride, J. T. Baker; Phosphoric Acid, J. T. Baker; Sodium Phosphate Monobasic, J. T. Baker; Potassium Phosphate, J. T. Baker; EDTA, Dihydrate, J. T. Baker; 2-hydroxypropyl-β-cyclodextrin, Alfa Aesar; Gamma-cyclodextrin (Cavamax W8); 2-hydroxyethyl-β-cyclodextrin, Sigma Aldrich; Heptakis 2,6-di-O-methyl-β-cyclodextrin, Sigma Aldrich; Sodium Sulfobutylether-β-cyclodextrin, Zibo Qianhui.

Stock Buffer Solutions: Stock buffer solutions, shown in Table 1, were prepared at 2-5 times the concentration of the pharmaceutical product by dissolving EDTA, Citric acid, Sodium Citrate, and Sodium Chloride in a clean/dry volumetric flask containing purified water. Each excipient was mixed until fully dissolved and the flask was diluted to volume with purified water.

TABLE 1
Stock Buffer Solutions

| | Concentration Range |
|---|---|
| Excipient | (mg/mL) |
| EDTA | 0.1-0.4 |
| Citric acid Anhydrous | 1.2-1.6 |
| Sodium Citrate | 1.5-2.1 |
| Dihydrate | |
| Sodium Chloride | 26.4-27.6 |

Preparation of Lab Batches: Tared a clean/dry beaker+stir bar. Weighed and transferred Budesonide and cyclodextrin into the beaker and mixed dry to achieve uniform dispersion. Dissolved with a portion of stock buffer solution to create a saturated ionic phase and mixed for 5-20 minutes. Slowly diluted with the buffer solution and mixed for an additional 5-10 minutes. The pH of the solution was then adjusted with 1M citric acid or 1M NaOH and diluted to the final volume with purified water. While mixing, the solution was sparged with nitrogen for 30 minutes.

A study was performed to evaluate the effect of 2-Hydroxypropyl-β-cyclodextrin (HP-β-CD) and pH on the formulation stability. Preliminary proof of concept studies had determined that <10% HP-β-CD was required with the new complexation process. The percent of HP-β-CD ranged from 4% to 8% and the effects on both the solubility and stability of Budesonide were evaluated. The pH tested ranged from 3.5 to 4.5. The pH range was chosen based on previous data collected during the forced degradation studies of the API.

The assay, impurities, pH, and osmolality were determined for time zero and separate accelerated stability studies were conducted to evaluate the complexation stability and degradation of the API in solution. The stability studies were carried out for 90 days at 2-8° C., 25° C., and 40° C. The results of the study demonstrated the process produced a cyclodextrin/Budesonide complex which is stable under all of the storage conditions studied.

There was no visible agglomeration or precipitation noted during this study. There were only 2 degradants that grew in the formulations: USP Impurity D and the unknown impurity which elutes at a relative retention time (RRT) of 0.35. All the observed values were below the ICH qualification of identification threshold of 1.0% for Impurity D and 0.5% for the unknown impurity.

Complexation Rate in Buffer Vs. Water:

To evaluate the initial association rate of Budesonide with 2-hydroxypropyl-beta-cyclodextrin, excess Budesonide was mixed with 2 g of 2-HY-B-CD in 12.5 mL of concentrated buffer solution or water. 1.0 mL of this slurry was filtered through a 0.2 syringe filter to remove un-complexed Budesonide and evaluated by UV-Vis at 2 minute intervals. A positive control prepared from filtered Budesonide Inhalation Suspension containing 0.25 mg/mL of Budesonide was used to ensure non-interference. Filtered samples of suspension showed that less than 1.2 μg/mL of Budesonide was not filtered from the solution phase. This is consistent with the particle size distribution of the API which has a small percentage of Budesonide with <0.2 μm particle size diameter. This small amount of Budesonide was determined not to be significant enough to bias the results.

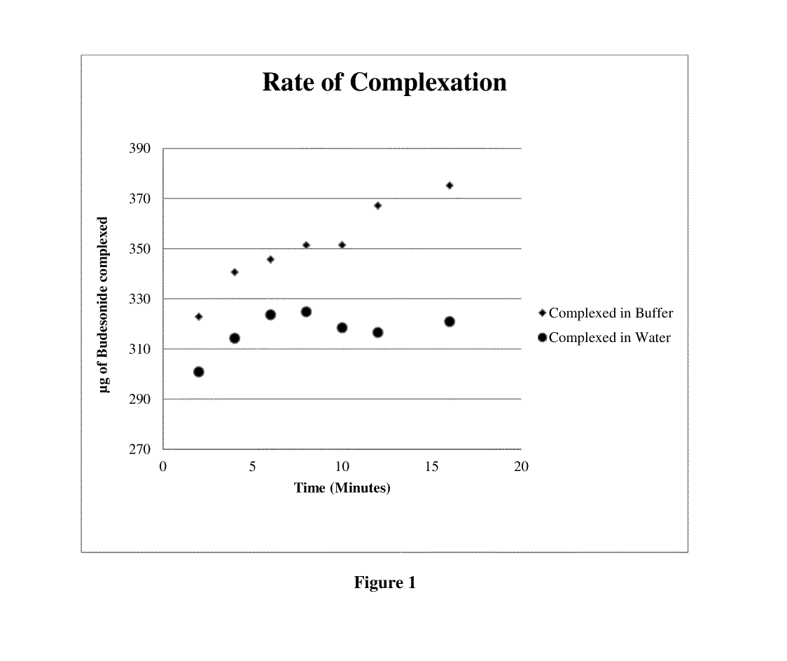

The results of the study, shown in FIG. 1, demonstrate that complexation between Budesonide and 2-hydoxypropyl-B-cyclodextrin occurs at a much higher rate in buffer than in water.

Complexation Efficiency in Buffer Vs. Water:

An end point analysis was conducted to evaluate the efficiency of the formulation procedure. Laboratory batches of Budesonide Inhalation Solution were prepared in 3% 2-HY-B-Cyclodextrin and assayed to evaluate the efficiency of the formulation procedure. During the second study, the rate of complexation between Budesonide and 2-HY-B-Cyclodextrin was evaluated in both Buffer and Water.

Six laboratory batches of Budesonide Inhalation Solution were prepared as described above, 3 each in buffer and water. The amount of Budesonide complexed was evaluated at 5 minutes, 8 minutes and 12 minutes for each solvent and compared to the expected concentration at the 100% level. The results are shown in Table 2.

TABLE 2
Complexation of Budesonide in Buffer vs. Water

| | % Assay at T = min. | | |
|---|---|---|---|
| Solvent | 5 minutes | 8 minutes | 12 minutes |
| Buffer | 91.1 | 96.3 | 99.7 |
| Water | 71.9 | 78.1 | 89.7 |

The batches prepared in the concentrated buffer solution showed approximately a 22% increase in the initial complexation efficiency over those prepared in water. From the data it was determined that preparations at these molar ratios would reach 100% complexation after approximately 9 minutes in buffer and 16.2 minutes in water, an increase in efficiency of 180%.

Alternative Salts or Complexation:

The ionic strength of the buffer described above was calculated to be approximately 508 mol/m⁻³. Solutions of three alternative salts (Sodium Chloride, Phosphate buffer, and Potassium Chloride) were prepared at the same ionic strength and used to formulate laboratory batches of budesonide inhalation solution according to the procedure described above. The results, depicted in Table 3, show that complexation can be achieved in less than 10 minutes using either a buffer or a single salt, so long as the ionic strength is maintained.

TABLE 3
Assay Results for Alternative Salts

| Salt | % Assay |
|---|---|
| Formulation Buffer | 99.7 |
| NaCl | 99.1 |
| Phosphate Buffer | 100.3 |
| KCl | 99.1 |

Minimum Concentration of Cyclodextrin:

Laboratory batches of budesonide inhalation Solution 0.188 mg/mL were prepared to determine the minimum concentration of 2-Hydroxypropyl-B-cyclodextrin required for an effective formulation. Each laboratory batch was prepared as per the procedure described above. The molar ratio of cyclodextrin to budesonide for each batch is shown in Table 4.

TABLE 4
Assay Results

| | Molar Ratio | |
|---|---|---|
| % Cyclodextrin | CD:Budesonide | Assay |
| 3.0 | 50.3 | 99.4 |
| 2.5 | 41.9 | 95.7 |
| 2.0 | 33.6 | 95.5 |
| 1.5 | 25.2 | 91.5 |
| 1.0 | 16.8 | 85.9 |
| 0.5 | 8.4 | 75.9 |

The results of the study showed that 3.0% 2-Hydroxypropyl-B-cyclodextrin, or a molar ratio of 50.33, may be used to achieve 100% complexation of Budesonide.

Both 2.5% and 2.0% cyclodextrin achieved high enough complexation efficiency to be effective. However, below 3.0% beta-CD, the ratio between complexed Epimers A and B of Budesonide was affected. The USP monograph includes criteria for Epimer A of Budesonide, which states it must be within 40%-51% of the total content. It was noted during this study that below 3.0% Beta-cyclodextrin, the ratio of epimer A exceeded 51%. For this reason it was determined that the minimum desirable concentration of cyclodextrin for the formulation to meet the USP monograph is NLT 3.0%.

Alternative Beta-Cyclodextrins for Complexation: Laboratory batches of budesonide inhalation solution 0.188 mg/mL were prepared in buffer and water according to the procedure described above using 2-hydroxyethyl-B-cyclodextrin and Heptakis 2,6-Di-O-Methyl-B-cyclodextrin.

2-Hydroxyethyl-B-cyclodextrin: 2-Hydroxyethyl-B-cyclodextrin is one of the weaker complexing vehicles in the beta class of cyclodextrins. This cyclodextrin required a higher concentration to achieve 100% complexation of the available budesonide than the 2-Hydroxypropyl derivative. But the data shows that 100% efficiency is possible within the proposed range of cyclodextrin (3%-8%) for the formulation. 2,6-di-O-Methyl-B-cyclodextrin: 2,6-di-O-Methyl-B-cyclodextrin is one of the strongest complexing vehicles in the beta class of cyclodextrins. 100% efficiency of complexation with available budesonide at 0.188 mg/mL was achieved in buffer between 3-5 minutes. See Table 5.

TABLE 5
% Assay for Alternative Beta-cyclodextrins

| β-Cyclodextrin | % Assay | % Cyclodextrin |
|---|---|---|
| 2-Hydroxyethyl | 66.7 | 3% |
| 2-Hydroxyethyl | 90.3 | 6% |
| 2-Hydroxyethyl | 96.4 | 8% |
| 2,6-di-o-mehtyl | 101.9 | 3% |
| Sulfobutyl-ether | 98.0 | 4% |

### Example 2

Filtration studies conducted on 5 laboratory batches have demonstrated the complexation procedure of the invention reaches virtually 100% efficiency in less than 10 minutes room temperature for the complexation of Budesonide with 2-hydroxypropyl-β-cyclodextrin and does not require high sheer forces or the use of organic solvents such as alcohol, propylene glycol, etc. In this study 50 mL of 5 separate laboratory batches were filtered through PTFE, PVDF, and PES 0.22 μm filters. In the case that the complexation reaction was not completed, the filtration process would remove the un-complexed budesonide producing a significant difference between the assay values before and after filtration. The results of the study are summarized in Table 6. For each of the lab batches tested, no significant difference between pre and post filter assay values was detected, indicating 100% complexation.

TABLE 6
Table 6: Summary of Filtration Study Assay Values

| | Batch | | | | | | | | | |
|---|---|---|---|---|---|---|---|---|---|---|
| | 1 | | 2 | | 3 | | 4 | | 5 | |
| Filter | Assay | Diff. | Assay | Diff. | Assay | Diff. | Assay | Diff. | Assay | Diff. |
| Pre-filter | 106.8 | NA | 105.3 | NA | 106.0 | NA | 106.2 | NA | 105.6 | NA |
| PES | 106.7 | 0.1 | 103.8 | 1.5 | 105.8 | 0.2 | 106.7 | −0.5 | 105.7 | −0.1 |
| PTFE | 106.8 | 0.0 | 104.8 | 0.5 | 105.9 | 0.1 | 105.9 | 0.3 | 105.9 | −0.3 |
| PVDF | 106.9 | −0.1 | 105.8 | −0.5 | 105.8 | 0.2 | 106.5 | −0.3 | 105.5 | 0.1 |

One particular formulation using 2-hydroxypropyl-β-cyclodextrin as the complexing agent has demonstrated both physical and chemical stability. The formulation contain Budesonide, 2-hydroxypropyl-β-cyclodextrin, Citric acid Anhydrous, Sodium Citrate Dihydrate, EDTA and Sodium Chloride.

EDTA and Gamma-Cyclodextrin Effects on the Formulation Stability.

Previous studies have shown that EDTA helps control the growth of impurity D. The amount of the EDTA needed was evaluated. γ-CD has been used as a stabilizing agent, approved by the FDA up to 5% for intravenous injection. A study was designed to evaluate the effects of EDTA and γ-CD on the formulation stability. Both γ-CD and EDTA were varied beginning at 0.05% and tested at 30 day intervals for 90 days stored at 40° C.

There was no visible agglomeration or precipitation. There were only 2 degradants that grew in the formulations: Impurity D and an unknown impurity which elutes at a relative retention time (RRT) of 0.35, which correlates to the previous studies. There was no change in Osmolality or pH over the period of the study. EDTA and γ-CD had a positive effect on stability. There were no significant effects observed when varying the amounts EDTA and γ-CD. All the observed values were below the threshold of 1.0% for Impurity D and 0.5% for the unknown impurity.

1. A method of preparing a pharmaceutical product comprising
forming an aqueous complexing solution having an osmolality of at least 400 mOsm/kg or an ionic strength of at least 290 mol/m⁻³ and containing cyclodextrin and budesonide, the cyclodextrin and budesonide capable of forming a cyclodextrin-budesonide inclusion complex,
permitting the cyclodextrin and budesonide inclusion complex to form, and
diluting the complexing solution to provide a pharmaceutical composition having an osmolality of between 260 mOsm/kg and 330 mOsm/kg.

2. The method of claim 1 wherein the osmolality of the complexing solution is at least: 400 mOsm/kg, 600 mOsm/kg, 900 mOsm/kg, 1200 mOsm/kg, 1500 mOsm/kg, 1800 mOsm/kg, 2100 mOsm/kg, 2400 mOsm/kg, 2700 mOsm/kg, 3000 mOsm/kg, or 3500 mOsm/kg

3. The method of claim 1 or 2, wherein the ionic strength of the complexing solution is at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³.

4. The method of any one of claims 1-3, wherein the molar ratio of cyclodextrin to budesonide in the complexing solution is between 20:1 and 100:1.

5. The method of claim 4, wherein the molar ratio of cyclodextrin to budesonide in the complexing solution is between 40:1 and 60:1

6. The method of any one of claims 1-5, wherein the complexing solution is a 60%-100% cyclodextrin saturated solution.

7. The method of claim 6, wherein the complexing solution is a 90%-100% cyclodextrin saturated solution.

8. The method of any one of claims 1-7 wherein the pH of the complexing solution is below 6, or between 3.5 and 4.5 and contains NaCl, a buffer and EDTA.

9. The method of any one of claims 1-8 wherein the aqueous complexing solution is formed by first mixing the cyclodextrin as a solid with the budesonide as a solid to form a mixture of solids, and then contacting the mixture of solids with an ionic aqueous solubilizing solution to form the complexing solution, wherein the ionic aqueous solubilizing solution has an ionic strength of at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³.

10. The method of claim 9, wherein the ionic aqueous solubilizing solution contains NaCl, a buffer and EDTA.

11. The method of any one of claims 1-7 and 9-10, wherein the complexing solution is contacted with a pH adjusting agent to adjust the pH of the complexing solution to between 3.5 and 4.5.

12. The method of any one of claims 1-11, wherein the cyclodextrin is 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

13. The method of claim 12, wherein the cyclodextrin is 2-hydoxypropyl-B-cyclodextrin.

14. A method of preparing a pharmaceutical product comprising
forming an aqueous complexing solution containing cyclodextrin and budesonide, the cyclodextrin and budesonide capable of forming cyclodextrin-budesonide inclusion complexes, wherein the molar ratio of cyclodextrin to budesonide in the complexing solution is greater than 40:1,
permitting the cyclodextrin and budesonide inclusion complex to form, and
diluting the complexing solution to provide a pharmaceutical composition, wherein the pharmaceutical composition has a pH of less than 6 or between 3.5 and 4.5, and an mOsm of between 260 and 330.

15. The method of claim 14, wherein the molar ratio of cyclodextrin to budesonide in the complexing solution is between 40:1 and 100:1.

16. The method of claim 14, wherein the molar ratio of cyclodextrin to budesonide in the complexing solution is between 40:1 and 60:1.

17. The method of any one of claims 14-16, wherein the osmolality of the complexing solution is at least: 400 mOsm/kg, 600 mOsm/kg, 900 mOsm/kg, 1200 mOsm/kg, 1500 mOsm/kg, 1800 mOsm/kg, 2100 mOsm/kg, 2400 mOsm/kg, 2700 mOsm/kg, 3000 mOsm/kg, or 3500 mOsm/kg.

18. The method of any one of claims 14-17 and 19, wherein the ionic strength of the complexing solution is at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³.

19. The method of any one of claims 14-16, wherein the osmolality of the complexing solution is between 400 mOsm/kg and 3500 mOsm/kg.

20. The method of any one of claims 14-17 and 19, wherein the ionic strength of the complexing solution is between 290 mol/m⁻³ and 1500 mol/m⁻³.

21. The method of any one of claims 14-20, wherein the complexing solution is a 60%-100% cyclodextrin saturated solution.

22. The method of claim 21, wherein the complexing solution is a 90%-100% cyclodextrin saturated solution.

23. The method of any one of claims 14-22 wherein the aqueous complexing solution is formed by first mixing the cyclodextrin as a solid with the budesonide as a solid to form a mixture of solids, and then contacting the mixture of solids with an aqueous solubilizing solution to form the complexing solution.

24. The method of claim 23, wherein the aqueous solubilizing solution is an ionic solution containing NaCl, a buffer and EDTA and wherein the ionic strength of the aqueous solubilizing solution is at least: 290 mol/m⁻³, 435 mol/m⁻³, 650 mol/m⁻³, 870 mol/m⁻³, 1090 mol/m⁻³, 1200 mol/m⁻³, 1400 mol/m⁻³, or 1500 mol/m⁻³.

25. The method of any one of claims 23-24, wherein the pH of less than 6 is provided by contacting the aqueous solubilizing solution with a pH adjusting agent to adjust the pH of the complexing solution to below 6, or to between 3.5 and 4.5.

26. The method of any one of claims 14-25, wherein the cyclodextrin is 2-hydoxypropyl-B-cyclodextrin, 2-hydroxyethyl-B-cyclodextrin, Heptakis 2,6-Di-O-Methyl-B-cyclodextrin, or sulfobutyl-ether cyclodextrin.

27. The method of claim 26, wherein the cyclodextrin is 2-hydoxypropyl-B-cyclodextrin.

28. The method of any one of claims 1-27, wherein the complexing solution is mixed for less than 120 minutes, or less than 60 minutes or less than 30 minutes to form said inclusion complexes, where at least 95%, at least 96%, at least 97%, at least 98%, or at least 99% of the budesonide in the complexing solution is part of an inclusion complex.

29. The method of any one of claims 1-28, wherein the budesonide is present in the complexing solution at a concentration of between 0.010 mg/mL and 7.5 mg/mL.

30. The method of any one of claims 1-29, wherein the budesonide is present in the pharmaceutical composition at a concentration of between 0.001 mg/mL and 0.75 mg/mL, of between 0.05 mg/mL and 0.60 mg/mL, of between 0.09 mg/mL and 0.50 mg/mL, or of between 0.10 mg/mL and 0.25 mg/mL.

31. The method of any one of claims 1-30, wherein the complexing solution is free of organic solvents.

32. The method of any one of claims 1-31, wherein the complexing solution and the pharmaceutical composition are free of preservatives other than EDTA and citric acid.

33. The method of any one of claims 1-32, wherein the complexing solution is free of any one of, any combination of, or all of thickening agents, molecules other than budesonide that form a complex with cyclodextrin (accompanying guest), and stabilizing polymers and the pharmaceutical composition is free of any one of, any combination of, or all of thickening agents, molecules other than budesonide that form a complex with cyclodextrin (accompanying guest), and stabilizing polymers.

34. A composition comprising an aqueous solution having an osmolality of at least 400 mOsm/kg or an ionic strength of at least 290 mol/m⁻³ and containing a cyclodextrin and budesonide, wherein at least 95%, at least 96%, at least 97%, at least 98%, or even at least 99% of the budesonide in the solution is complexed with the cyclodextrin, and wherein the aqueous solution is free of a co-solvent.

35. The composition of claim 34, wherein the molar ratio of cyclodextrin to budesonide is at least 40:1, at least 45:1, at least 50:1, at least 55:1, at least 60:1, at least 75:1 or between 40:1 and 100:1.

36. The composition of any one of claims 34-35, wherein the osmolality is between 400 mOsm/kg and 3500 mOsm/kg.

37. The composition of any one of claims 34-36, wherein the ionic strength is between 350 mol/m⁻³ and 1500 mol/m⁻³

38. The composition of any one of claims 34-37, wherein the solution is free of any one of, any combination of, or all of (i) thickening agents, (ii) molecules other than budesonide that form a complex with cyclodextrin (accompanying guest), and (iv) stabilizing polymers.

39. A composition comprising a dry mixture of a cyclodextrin and budesonide, wherein the molar ratio of cyclodextrin to budesonide is at least 40:1, at least 45:1, at least 50:1, at least 55:1, at least 60:1, at least 75:1, or between 40:1 and 100:1.

40. A pharmaceutical composition comprising an aqueous solution having an osmolality of between 260 and 330, wherein the solution contains cyclodextrin and budesonide in molar ratio of at least wherein the molar ratio of cyclodextrin to budesonide is at least 40:1, at least 45:1, at least 50:1, at least 55:1, at least 60:1, at least 75:1, or between 40:1 and 100:1, wherein the budesonide is present in a concentration of between of between 0.001 mg/mL and 0.75 mg/mL, of between 0.05 mg/mL and 0.60 mg/mL, of between 0.09 mg/mL and 0.50 mg/mL, or of between 0.10 mg/mL and 0.25 mg/mL, and wherein at least 95% of the budesonide in the solution is complexed with cyclodextrin, and EDTA.

41. The pharmaceutical composition of claim 40, wherein the aqueous solution is a buffered aqueous solution.

42. The pharmaceutical composition of claim 40 or 41, wherein the aqueous solution further comprises a citrate buffer, and sodium chloride.

43. The pharmaceutical composition of any one of claims 40-42, wherein the aqueous solution is free of any one of, combination of, or all of (i) a co-solvent, (ii) sodium benzoate, (iii) any preservative other than citric acid and EDTA, (iv) a thickening agent, (v) molecules other than budesonide that form a complex with cyclodextrin (accompanying guest), and (vi) stabilizing polymers.

44. A pharmaceutical composition consisting of a cyclodextrin, budesonide, NaCl, EDTA, a buffer and water.

45. The pharmaceutical composition of claim 44, wherein the osmolality of the pharmaceutical composition is between 260 mOsm/kg and 330 mOsm/kg.

46. The pharmaceutical composition of claim 44 or 45, wherein the molar ratio of cyclodextrin to budesonide is at least 40:1, at least 45:1, at least 50:1, at least 55:1, at least 60:1, at least 75:1, or between 40:1 and 100:1.

47. The pharmaceutical composition of any one of claims 44-46, wherein the budesonide is present in a concentration of between of between 00.001 mg/mL and 0.75 mg/mL, of between 0.05 mg/mL and 0.60 mg/mL, of between 0.09 mg/mL and 0.50 mg/mL, or of between 0.10 mg/mL and 0.25 mg/mL.

48. The pharmaceutical composition of any one of claims 44-47, wherein at least 95%, at least 96%, at least 97%, at least 98%, or at least 99% of the budesonide in the solution is complexed with cyclodextrin.

49. A pharmaceutical product prepared by any one of claims 1-33.
